(19) United States
(12) Patent Application Publication
(10) Pub. No.: US 2019/0247140 A1
(43) Pub. Date: Aug. 15, 2019

(54) DISPOSABLE MATERIAL TO PROTECT NON-TARGET TISSUE DURING TREATMENT OF THE SKIN AND METHOD OF PROTECTING THE SKIN

(71) Applicant: Eric F. BERNSTEIN, Gladwyne, PA (US)

(72) Inventor: Eric F. BERNSTEIN, Gladwyne, PA (US)

(21) Appl. No.: 16/271,512

(22) Filed: Feb. 8, 2019

Related U.S. Application Data
(60) Provisional application No. 62/628,524, filed on Feb. 9, 2018.

(51) Int. Cl. A61B 90/00; A61N 5/06; A61L 31/10; A61L 31/14; A61B 46/00

(52) U.S. Cl. CPC A61B 90/04; A61N 5/0616; A61L 31/10; A61L 31/14; A61B 46/40; A61B 2090/049; A61N 2005/067; A61N 2005/0654; A61N 2005/0655; A61N 2005/0659; A61N 2005/0661; A61N 2005/0662; A61N 2005/0651

(57) ABSTRACT

A disposable material, pre-cut to various shapes or able to be cut or shaped at the time of use, to protect non-target tissue, excluding the eyes, during treatment of the skin is disclosed. The disposable material comprises a flexible material comprising at least one first layer comprising a material that mitigates the transmission of electromagnetic radiation from an radiation source; a backing layer that is located on top of the first layer and is configured to diffusively reflect the electromagnetic radiation without direct specular reflection; and an adhesive for removably attaching the disposable material to the skin. There is also disclosed a method of protecting non-target tissue, excluding the eyes, by applying the disclosed material to a desired location.

This application claims the benefit of priority to U.S. Provisional Application No. 62/628,524 filed Feb. 9, 2018, which is incorporated herein by reference in its entirety.

### FIELD OF USE

The present disclosure generally relates to a disposable material, such as a patch, to cover and protect non-target tissue during a procedure with a light or other energy-based device, such as a laser, light emitting diode (LED), or intense pulsed light (IPL) treatment. The present disclosure also relates to a method of using the disposable material to protect non-target tissue.

### BACKGROUND

Various laser, light, LED, and other energy-based device treatments have been widely used by consumers, estheticians, dermatologists, and other physicians in spas, at home, and in physician offices. Such devices have been used to rejuvenate, repair, or resurface skin, remove freckles and other benign pigmented lesions, treat pre-cancers and skin cancers, psoriasis, eczema and other skin conditions, remove vascular lesions such as rosacea, spider veins and port-wine stains, remove tattoos, remove unwanted hair, and many more uses. For example, lasers and other energy-based devices have been used to treat the five key signs of skin aging including: fine lines and wrinkles, enlarged pores, sagging skin, pigment abnormalities like freckles, and spider veins with generalized redness and flushing, as well as skin cancer.

The objective of laser and other energy-based device treatments on the skin is to remove or rejuvenate target tissues while sparing non-target tissues that may surround target tissues or be intermingled with target tissues. In addition to treating the foregoing signs of aging, laser hair removal, either temporary or permanent, is among the most-administered laser treatments and targets the pigment in hair follicles to stimulate inflammation that induces hair regression. This treatment has the simultaneous objective of sparing normal skin including: nevi or moles, tattoos, pigmented or tanned skin, tattoos, non-target hair, and birthmarks. Incidental treatment of moles or nevi, lentigos, seborrheic keratosis, congenital nevi, freckles, café au lait macules and other unintended targets can cause skin injury, damage or alter the appearance of these lesions making further examination more challenging.

The disclosed disposable material comprising a flexible material is directed to overcoming one or more of the problems set forth above and/or other problems of the prior art.

### SUMMARY

There is disclosed a disposable material to protect non-target tissue during treatment of the skin. In an embodiment, the disposable material comprises a flexible material comprising at least one first layer comprising a material that mitigates the transmission of electromagnetic radiation from an radiation source; a backing layer that is located on top of the first layer and is configured to diffusively reflect the electromagnetic radiation without direct specular reflection; and an adhesive for removably attaching the disposable material to the skin.

There is also disclosed a method to protect non-target tissue from electromagnetic radiation during treatment of an adjacent portion of the patient's skin. In an embodiment, the method comprises adhering to the non-target tissue prior to treatment, a disposable material comprising a flexible material. In different embodiments, this flexible material is non-metallic, or includes a metal layer for covering and protecting non-target tissue. In an embodiment, the material comprises at least one first layer comprising material that mitigates the transmission of electromagnetic radiation from an radiation source; a backing layer that is located on top of the first layer and is configured to diffusively reflect the electromagnetic radiation without direct specular reflection; and an adhesive sufficient to removably adhere the disposable material to the non-target tissue.

### DETAILED DESCRIPTION

In the simplest embodiment, there is described a disposable material and method for covering tissue that should not be exposed to electromagnetic radiation during a skin treatment, such as laser surgery, with a disposable material.

As used herein, “tissue” is intended to mean the thin layer of skin forming the natural outer covering of the body of a person or animal but it excludes the eyelids.

As used herein, “non-target tissue” is intended to include congenital nevi, acquired nevi, non-target non-lesional skin, such as during psoriasis treatment or tattoo treatment, lentigos, freckles, café au lait macules, seborrheic keratosis, actinic keratosis, eyebrows and other hair-bearing sites, decorative tattoos, as well as cosmetic tattoos, such as lip liner, eyebrows, nipple tattoos, and camouflage tattoos.

As used herein, “non-target tissue” does not include the eyes. Thus, the disclosed disposable materials are not intended to cover or protect the eyes from exposure to electromagnetic radiation during a skin treatment.

Depending on the end use, the disposable materials described herein may be pre-cut or be in the form of a sheet or roll that can be cut to fit any size immediately prior to treatment.

In one embodiment, there is described disposable material comprising at least one first layer for contacting the skin that comprises a metallic material, non-metallic material, or both that mitigates the transmission there-through of electromagnetic radiation, such as laser light. When desired, the first layer may comprise at least one metallic layer to help stop or mitigate the transmission of electromagnetic radiation, such as laser light. In this embodiment, the at least one metallic layer may comprise aluminum, copper, brass, tin, steel, or combinations or alloys thereof.

The metallic layer may be in the form of a metallized polymer or a foil, and may be in a thickness needed to prevent the transmission of electromagnetic radiation, such as a thickness ranging from 0.001 to 1 mm. In various embodiments, the upper thickness may have any increment of 0.1 mm, within the broader range of 0.001 to 1 mm, such as 0.9 mm, 0.8 mm, 0.7 mm, 0.6 mm, 0.5 mm, etc. Likewise, the lower thickness may increase in increments of 0.001 mm within the broader range of 0.001 to 1 mm, such as 0.002 mm, 0.003 mm, 0.004 mm, etc.

The first layer can further be made of light or white-colored material, or having a finish to reflect the light away diffusely so as not to cause a risk of direct specular reflection, such as a matte finish. In this embodiment, the surface of the white-colored material or the matte finish, or a white-colored matte finish, safely diffuses the refection of light impinging on the surface. For example, the first layer backing can be made of coated paper, non-latex synthetic rubber, plastic, or combinations thereof.

The disposable material described herein may further comprise an adhesive for removably attaching the disposable material to the skin. In one embodiment, the adhesive is located on the first layer such that it is in direct contact with the non-target tissue, excluding the eyes, and is covered by the first layer. Non-limiting examples of the adhesive described herein include a vinyl resin, such as an acrylate chosen from methacrylates and diacrylates.

In another embodiment, the adhesive is not located on the first layer, but is located on the backing layer. The backing layer is typically larger than the first layer and comprises the adhesive around its periphery. In this embodiment, the adhesive is in direct contact with the skin but not the non-target tissue.

In one embodiment, the first layer comprises a fabric, paper, plastic material, or rubber material, or combinations thereof. For example, the at least one first layer may comprise acrylic, non-latex rubbers, wax-coated paper, or combinations thereof.

In an embodiment, the first layer may further comprise a foam or pad layer. In an embodiment, the foam or pad may comprise an absorbent pad, such as one made of cotton, alone, or with a thin, porous-polymer coating over the pad, to keep it from sticking to the non-target tissue. The pad may also be medicated with an antiseptic solution. In some embodiments, the pad is made of a water-absorbing hydrogel.

In an embodiment, the backing layer comprises a woven fabric, a plastic material chosen from PVC, polyethylene or polyurethane, or a rubber material, wherein the backing layer further comprises a matte finish to diffuse electromagnetic radiation. The backing layer may comprise a biocompatible foamed plastic material.

In an embodiment, the disposable material described herein has a total thickness ranging from 0.1 mm to 10 mm, such as from 0.3 mm to 7.0 mm, 0.5 mm to 5 mm, or even 0.75 to 2.5 mm.

In an embodiment, the disposable material may be in the form of a sheet or roll. The sheet or roll may be pre-cut or perforated, so as to comprise patches of pre-cut sizes and shapes. For example, the pre-cut patch has a circular shape with a diameter ranging from 1 mm to 80 mm, such as from 2 mm to 60 mm, or even 5 mm to 50 mm. In an embodiment, the pre-cut patch may have an oval shape with the major diameter ranging from 5 mm to 100 mm, such as 10 mm to 80 mm, or even 15 mm to 60 mm. Whether in the form of a circle or oval, the pre-cut patch may further include a non-adhesive tab to assist in applying the patch to the skin, such as for grasping the material prior to application.

In another embodiment, the disposable material may be in the form of a sheet or roll that is not pre-cut or perforated. Rather, in this embodiment, the disposable material is in the form of a sheet or tape that can be cut into desired shapes and sizes, such as by a distributor or the end user.

In an embodiment, the disposable material can be used to protect non-target tissue, excluding the eyes, from electromagnetic radiation having a wavelength ranging from 200 nm to 11,000 nm. Devices that can be used to emit such radiation include a laser, light emitting diode, incandescent bulb, UV lamp, or intense pulsed light (IPL).

The material or patch described herein can be used to cover various non-targeting areas of the skin, excluding the eyes. Non-limiting examples of the types of light and/or laser treatments include excimer/psoriasis lasers that include UVB 290-320 nm, also UVA 320-400 nm. This can be laser or non-laser light administered from fluorescent tube lighting or LEDs.

In an embodiment, the method can be used to protect the skin, excluding the eyes, from exposure to a long-pulse duration, 532 nm vascular laser. Potassium Titanyle Phosphate (KTiOPO₄) or “KTP” is a non-linear optical material which is commonly used for frequency doubling diode pumped solid-state lasers such as Neodymium Yttrium-Aluminum-Garnet Laser (Nd:YAG) and other neodymium-doped lasers. KTP frequency-doubling crystals generating green light from 1,064 Nd:YAG lasers, Nd:YAG lasers emitting 1,064 nm radiation; long pulse-duration alexandrite lasers emitting 755 nm laser light; and 585 nm and 595 nm light from pulsed-dye lasers that are used for treatment of vascular lesions including but not limited to spider veins, diffuse redness, rosacea, poikiloderma of Civatte, sun-damage, port-wine stain birthmarks, cherry angiomas and nevus araneuses.

In an embodiment, the method can be used to protect the skin from exposure to a Q-switched nanosecond-domain and picosecond-domain lasers emitting 532 nm, 694 nm (ruby laser), 785 nm Ti:Sapphire, 755 nm alexandrite laser, 1,064 nm, and dye cartridges used with Q-switched lasers converting energy to 585 nm and 659 nm. These lasers are typically used to remove decorative or cosmetic tattoos and well as pigmented lesions such as ephilides, lentigos, keratoses, café au lait macules, seborrheic keratoses, or melasma.

In an embodiment, the method can be used to protect the skin, excluding the eyes, from exposure to an Erbium Yttrium Aluminum Garnet Laser [Er:YAG] (2940 nm). This pulsed laser can remove skin layers of a thickness of 5 to 15 μm, and thus is particularly useful for cosmetic treatments.

In an embodiment, the method can be used to protect the skin, excluding the eyes, from exposure to a laser chosen from solid state, diode and fiber lasers emitting at 1320 nm, 1440 nm, 1550 nm, and 1,720 nm, respectively, which are typically used to treat photoaging including but not limited to fine lines and wrinkles, enlarged pores, sagging skin, as well as acne scarring or other scars.

In an embodiment, the method can be used to protect the skin, excluding the eyes, from exposure to a CO₂ laser emitting at 10,600 nm. The radiation of this laser, when used in a pulsed or scanned mode, is ideal for skin resurfacing. When using this laser, the epidermal (Stratum corneum and epidermis) and uppermost dermal layers can purposefully and extensively be removed. Besides the removal of the epidermis, the residual heat stimulates dermal remodeling that provokes a remodeling of connective tissue as well a new synthesis of collagen. The re-growing skin is improved in appearance due to a reduction of wrinkles and scars.

In another embodiment, the method can be used to protect the skin, excluding the eyes, from exposure to long pulse-duration hair removal lasers emitting at 755 nm (alexandrite laser), 810 nm and 1,060 nm (diode laser), 1,064 nm (Nd:YAG laser), and 694 nm (ruby laser). In general, the Nd:YAG laser provides the highest penetration depth and produces photothermolysis of the hair shaft with subsequent inflammation that induces a resting phase in terminal hairs.

In additional embodiments, there is disclosed intense pulsed light (IPL) which emits a broad spectrum light with cut-off filters starting in the green at 510 nm and going into the red at 660 nm and many in-between and extending into the infrared at 2000+ nm and is typically used for treatment of pigmented lesions such as ephilides, lentigos, keratoses, café au lait macules, seborrheic keratoses, or melasma; vascular lesions such as spider veins, diffuse redness, rosacea, poikiloderma of Civatte, sun-damage, port-wine stain birthmarks, cherry angiomas and nevus araneuses; laser hair removal; and rejuvenation of sun-damaged skin including the 5 key signs of skin aging, which are fine lines and wrinkles, enlarged pores sagging skin and pigmentation.

The disclosed disposable material can also be used to protect non-targeted tissue, excluding the eyes, during photodynamic therapy (PDT) with aminolevulinic acid HCl (ALA). Commercially, there are a number of treatment options including Levulan® Kerastick® or ALA (aminolevulinic acid HCl) for Topical Solution, 20% (Levulan Kerastick) plus blue light illumination using the BLU-U® Blue Light Photodynamic Therapy Illuminator (Levulan PDT) for the treatment of actinic keratosis of the face, scalp or body. Actinic keratosis (AKs), are rough-textured, dry, scaly patches on the skin that can lead to skin cancer.

Photodynamic therapy (PDT) with ALA is a two-part treatment that uses a light activated drug therapy to destroy AKs. In this embodiment, a topical solution is applied to the full area of concern for a time sufficient to allow the solution to be absorbed by the AK cells where it is converted to a chemical that makes the cells extremely sensitive to light. When the AK cells are exposed to the BLU-U Blue Light Illuminator or Broad Band Light, or other light sources such as IPLs, lasers, light emitting diodes, a reaction occurs which destroys the abnormal cells in the AK. Other dyes or sensitizers such as dihematoporphyrin ether (DHE), benzaporphyrin derivative (BPD), purpurins, or phthalocyanines can be used in combination with fluorescent light sources, LEDs, lasers, and IPLs may also be used in a similar manner. In one embodiment, the disclosed disposable material could be used in the form of even larger non-adhesive cloth-type material to cover large portions of the body or face.

There is also disclosed a method to protect non-target tissue, excluding eyes, from electromagnetic radiation during treatment of an adjacent portion of the patient's skin by using the disclosed disposable material. In an embodiment, the method comprises adhering to the non-target tissue prior to treatment, a disposable material as described herein. For example, the disposable material that can be used in the described method comprises a flexible, non-metallic material comprising at least one first layer comprising a non-metallic material that mitigates the transmission of electromagnetic radiation from an radiation source; a backing layer that is located on top of the first layer and is configured to diffusively reflect the electromagnetic radiation without direct specular reflection, and an adhesive sufficient to removably adhere the disposable material to the non-target tissue.

In one embodiment, the method described herein includes placing a pre-cut patch over an area of the skin, excluding the eyes, to be protected. The pre-cut patch can have a thickness ranging from 0.1 mm to 10 mm, such as from 0.3 mm to 7.0 mm, 0.5 mm to 5 mm, or even 0.75 to 2.5 mm.

In an embodiment, the method described herein includes applying disposable material over non-target tissue, such as a mole, freckle, skin tag, lentigo, seborrheic keratosis, tattoo, decorative tattoo, cosmetic tattoo, hair bearing area, or combinations thereof. The disposable material may be in the form of a sheet or roll, which may comprise pre-cut sizes and shapes. For example, the pre-cut patches that may be used in the described method has a circular shape with a diameter ranging from 1 mm to 80 mm, such as from 2 mm to 60 mm, or even 5 mm to 50 mm. In another embodiment, the pre-cut patch may have an oval shape with the major diameter ranging from 5 mm to 100 mm, such as 10 mm to 80 mm, or even 15 mm to 60 mm. Whether in the form of a circle or oval, the pre-cut patch may further include a non-adhesive tab to assist in applying the patch to the skin, such as for grasping the material prior to application. In one embodiment, the disclosed material can be used for covering eyebrows, lips and other curved areas that contain non-target hair and cosmetic tattoos.

The method described herein may be use to apply the disposable material described herein over non-target tissue comprising a mole, freckle, skin tag, lentigo, seborrheic keratosis, tattoo, hair bearing area, or combinations thereof.

It will be apparent to those skilled in the art that various modifications and variations can be used as described herein without departing from the scope of the disclosure. Alternative implementations will be apparent to those skilled in the art from consideration of the specification and practice disclosed herein. It is intended that the specification and examples be considered as exemplary only, with a true scope of the disclosure being indicated by the following claims and their equivalents.

What is claimed is:

1. A disposable material to protect non-target tissue, excluding the eyes, during a treatment of skin, the disposable material comprising:
a flexible material comprising:
at least one first layer comprising a material that mitigates a transmission of electromagnetic radiation from an radiation source;
a backing layer that is located on top of the first layer and is configured to diffusively reflect the electromagnetic radiation without direct specular reflection; and
an adhesive for removably attaching the disposable material to the skin.

2. The disposable material of claim 1, wherein the adhesive is located on the first layer such that it is in direct contact with the non-target tissue and is covered by the first layer.

3. The disposable material of claim 1, wherein the backing layer is larger than the first layer and comprises the adhesive around its periphery, wherein the adhesive is in direct contact with the skin but not the non-target tissue.

4. The disposable material of claim 1, wherein the adhesive comprises a vinyl resin comprising an acrylate chosen from methacrylates and diacrylates.

5. The disposable material of claim 1, wherein the at least one first layer comprises a fabric, paper, plastic material, or rubber material, non-latex rubber, or combinations thereof.

6. The disposable material of claim 5, wherein the at least one first layer comprises acrylic, non-latex rubbers, wax coated paper, or combinations thereof.

7. The disposable material of claim 1, wherein the at least one first layer is metal free.

8. The disposable material of claim 1, wherein the at least one first layer comprises at least one metallic layer comprising aluminum, copper, brass, tin, steel, or combination or alloys thereof.

9. The disposable material of claim 1, wherein the backing layer comprises a woven fabric, a plastic material chosen from PVC, polyethylene or polyurethane, or a rubber material, wherein said backing layer further comprises a matte finish to diffuse said electromagnetic radiation.

10. The disposable material of claim 1, wherein the material has a thickness ranging from 0.5 mm to about 5 mm.

11. The disposable material of claim 1, wherein the electromagnetic radiation has a wavelength ranging from 200 nm to 11,000 nm, and the radiation source comprises a laser, light emitting diode, incandescent bulb, fluorescent lamp, UV lamp, or IPL.

12. The disposable material of claim 1, wherein the disposable material further comprising a non-adhesive tab for grasping said material prior to application.

13. The disposable material of claim 1, wherein the disposable material is in the form of a sheet or roll.

14. The disposable material of claim 13, wherein the sheet or roll comprises patches of a pre-cut size and shape.

15. The disposable material of claim 14, wherein the disposable material is a pre-cut patch having a circular shape with a diameter ranging from 2 mm to 60 mm, or an oval shape with a major diameter ranging from 10 mm to 80 mm.

16. A method to protect non-target tissue from electromagnetic radiation during treatment of an adjacent portion of the tissue, excluding the eye, the method comprising:
adhering to the non-target tissue prior to treatment, a disposable material comprising:
a flexible material comprising:
at least one first layer comprising a material that mitigates the transmission of electromagnetic radiation from an radiation source;
a backing layer that is located on top of the first layer and is configured to diffusively reflect the electromagnetic radiation without direct specular reflection; and
an adhesive sufficient to removably adhere the disposable material to the non-target tissue.

17. The method of claim 16, wherein treatment of an adjacent portion of the patient's skin comprises treating said skin with an electromagnetic radiation having a wavelength ranging from 200 nm to 11,000 nm.

18. The method of claim 16, wherein said treatment comprises the application of radiation to the skin from a laser, light emitting diode, incandescent bulb, fluorescent lamp, UV lamp, or IPL.

19. The method of claim 16, wherein said removably adhering comprises removably attaching a patch that is: cut to a desired shape and size from a sheet or roll; or that is a pre-cut patch having a circular shape with a diameter ranging from 2 mm to 60 mm, or an oval shape with a major diameter ranging from 10 mm to 80 mm, or combinations thereof.

20. The method of claim 16, further comprising cutting the disposable material to a desired shape and size prior to adhering it to the non-target tissue comprising a mole, freckle, skin tag, lentigo, seborrheic keratosis, tattoo, hair follicle, or combinations thereof.
